(19) United States
(12) Patent Application Publication
(10) Pub. No.: US 2025/0204922 A1
(43) Pub. Date: Jun. 26, 2025

(54) AN ENDOVASCULAR OCCLUSION DEVICE

(71) Applicant: University College Dublin, National University of Ireland, Belfield, Dublin (IE)

(72) Inventors: Cormac Farrelly, Dublin (IE); Eoin O'Cearbhaill, Dublin (IE); Sajjad Soleimaniamiri, Dublin (IE); Tom Fitzmaurice, Dublin (IE); Wenting Shu, Dublin (IE)

(21) Appl. No.: 18/850,158

(22) PCT Filed: Aug. 11, 2023
(86) PCT No.: PCT/EP2023/072331

§ 371 Date: Sep. 24, 2024

(30) Foreign Application Priority Data
Aug. 13, 2022 (GB) 2211862.4

(51) Int. Cl. A61B 17/12; A61B 17/00

(52) U.S. Cl. CPC A61B 17/12172; A61B 17/12109; A61B 2017/00477; A61B 2017/00862

(57) ABSTRACT

The present invention is concerned with an endovascular occlusion device for use in occluding a lumen of the endovascular system to treat certain medical conditions such as vascular malformations, bleeding vessels or occluding vessels in order to induce tissue ischemia or necrosis, the occlusion device including a deformable sleeve displaceable between a collapsed state and an expanded state and a conical array of cantilevered arms extending from a distal end of the sleeve and converging towards distal tips such as to define a first haemostatic valve about the distal end of the sleeve. The distal end of the sleeve will be at least partially covered with a polymer fluid impermeable membrane in order to provide a seal to prevent fluid flow through the occlusion device. The internal valve will be haemostatic regardless of the initial direction of blood flow and will allow distal endovascular delivery of a surgical instrument such as a catheter that can be manipulated and used to deliver therapeutic agents such as liquid or small particle embolics.

### FIELD OF THE INVENTION

The present invention is concerned with an endovascular occlusion device, in particular for use in occluding a lumen of the endovascular system to treat certain medical conditions and, more particularly, relates to an occlusion device for treating vascular malformations, bleeding vessels or occluding vessels in order to induce tissue ischemia or necrosis.

### BACKGROUND OF THE INVENTION

There are numerous clinical situations where blood vessels such as arteries and veins need to be occluded. Inducing necrosis or tissue ischaemia is a desired effect in certain medical conditions for example uterine fibroids, prostate hyperplasia, benign or malignant renal tumours and liver parenchyma when trying to induce compensatory hyperplasia in the untreated non-embolised liver in the weeks prior to liver surgery. The indications and number of procedures being performed with the aim of distal embolization in order to induce ischaemia and necrosis are growing.

There are also certain cancer treatments that require drug delivery into arteries and veins. Occluding flow proximally can redirect flow from distal vessels (flow redistribution) towards tumours. If drug delivery then occurs from the occluded proximal artery, for example in trans arterial chemoembolization (TACE) when delivered through an occlusion catheter (bTACE), more distal and complete penetration of drug delivery can be achieved.

Endoluminal occlusion has been performed for over sixty years. Over recent decades, major technological advances have improved the techniques used and different devices have been developed for different conditions and increasing indications. Early embolization materials included glass particles, hot contrast, paraffin, fibrin, and tissue fragments such as muscle fibres and blood clots. Present day occlusion materials include metallic devices, small particles, and liquid materials, which can be indicated for proximal or distal occlusion, high-flow and low-flow situations, and in large-calibre and small-calibre vessels, based on need.

METALLIC DEVICES: Coils are one of the most often applied embolization devices. They consist of spirals of different materials, such as stainless steel, platinum, alloys of these or other materials, and may be covered/coated by synthetic material to promote coagulation. One of the major concerns of coils is their proximal occlusion. If a coil is used in a bleeding condition, proximal occlusion may impede access to the bleeding spot in the event of an existing distal vascular network. Coil sizing is essential because coils that are too small can migrate, and oversized coils elongate and may therefore not occlude the vessel sufficiently. Oversized coils are also prone to dislodge and lead to non-target embolization.

An Amplatzer® vascular plug (AVP, AGA Medical, Golden Valley, MN, USA) is a braided Nitinol device that can be used to achieve permanent vessel occlusion. It is one type of embolization plug. A major advantage is that the vascular plug can be recaptured and repositioned. This allows a precise placement, with low risk of incorrect positioning or migration. In case of incorrect sizing, the plug can be withdrawn and substituted. However one major disadvantage of all such plugs is their dependence on the patient's effective coagulation. In case of severe coagulation disorders, occlusion may not be achieved by a plug. Occlusion time may also be unpredictable in the event of large vessel size and high-flow situations. Distal small vessel embolization distal to a deployed endovascular plug cannot be ensured and the plug although safe, quick and easy to deploy cannot induce distal small vessel ischaemia.

Coils and vascular occlusion plugs do not allow distal small vessel embolization and therefore are not appropriate to use alone if ischemia or necrosis are required.

LIQUID EMBOLICS: Liquid embolics are increasing in popularity as they are quick to use, leading to decreased radiation doses and they allow distal small vessel occlusion and are therefore good at inducing ischaemia and necrosis. Onyx® is a non-adhesive liquid embolization material that consists of three primary elements: a copolymer of ethylene and vinyl alcohol (EVOH) that is responsible for occlusion, dimethyl sulfoxide (DMSO) as a dissolvent, and tantalum powder for radiopaque visualization. N-butyl cyanoacrylate (NBCA) on the other hand is a fast embolization agent; therefore, radiation exposure might be reduced during intervention. One of the main disadvantages is that reflux from too hasty injection or inappropriate injection volumes may cause non-target embolization. Migration of the solidified material can also occur when retracting the catheter due to stripping the material attached to the catheter tip. Other causes of migration include premature polymerization and an inadequate relationship between polymerization speed and blood flow volume in high-flow vascular malformations.

SMALL PARTICLE EMBOLICS: Polyvinyl alcohol (PVA) is a synthetic, non-reabsorbable embolic agent for permanent vessel occlusion. Acrylic microspheres (Trisacryl gelatine) microspheres are non-reabsorbable microspheres with a precisely calibrated particle size and uniform shape. Their smooth surface structure prevents the microparticles from agglomerating, which permits better penetration of the particles to the distal arteries. Both these agents are good at inducing distal tissue necrosis but are dependent on the direction of blood flow to allow safe embolization and are prone to reflux when flow slows within the administered blood vessel.

Current vascular occlusion plugs and coils do not allow catheters to be placed distally for further embolization. There are an increasing number of clinical scenarios where it is recognized that distal small vessel embolization to the level of the capillary bed is required. This includes hepatic and portal vein embolization (used to induce hypertrophy of the contralateral liver lobe prior to surgery), embolization of bleeding varices in portal hypertension (entire length of varix requires occlusion rather than occlusion to the capillary bed), transarterial chemoembolization and prostate artery embolization. If liquids or small particles are used for embolization and to induce ischaemia the risk is non-target embolization and severe patient complications. Also, if concentrated drugs or particles that release radiation therapy (e.g., Yttrium90) are required to be given intravascularly for loco-regional therapy then the risk of reflux or non-target embolization can mean that suboptimal doses are delivered.

In some situations, it is not possible to safely use liquid or small particles. For example, if hepatic vein embolization is being performed distal vein embolization is essential to stop venous-venous collaterals developing as this can render the procedure ineffective. However, a catheter placed in the hepatic vein from jugular or femoral access cannot be used to safely infuse liquid or small particles as these agents would immediately follow the direction of blood flow out of the liver and into the heart and lungs.

An occlusion balloon catheter could be used to temporarily occlude the hepatic vein and allow liquid or small particle infusion through its central lumen but once the balloon is deflated the liquid embolic could then displace into the heart. Also, the occlusion balloon can get stuck in the liquid embolic.

Similarly, if liquid embolic or small particles are used in arteries or the portal vein once stagnant or slow flow is achieved there is increased risk of reflux of the liquid embolic, small particles or drug into another vessel and resultant non-target embolization/treatment.

It is therefore an object of the present invention to provide an improved occlusion device to address the above mentioned problems.

### SUMMARY OF THE INVENTION

According to the present invention there is provided an endovascular occlusion device comprising a deformable sleeve displaceable between a collapsed state and an expanded state; and a conical array of cantilevered arms extending from a distal end of the sleeve and converging towards distal tips such as to define a first haemostatic valve about the distal end of the sleeve.

Preferably, the resilience of the array of cantilevered arms acts to bias the first haemostatic valve into a closed state.

Preferably, the array of cantilevered arms are sufficiently resiliently deformable to permit the tips to separate radially to facilitate the passage of a surgical implement.

Preferably, the array of cantilevered arms are sufficiently resiliently deformable to exert a radially compressive force on a surgical implement projecting through the first haemostatic valve sufficient to maintain haemostasis between the implement and the first haemostatic valve.

Preferably, one or more of the cantilevered arms vary in cross sectional area along the length of the cantilevered arm.

Preferably, one or more of the cantilevered arms comprises a live hinge.

Preferably, the distal tips of the cantilevered arms are configured to cooperate with one another in order to establish a haemostatic seal at the distal tips.

Preferably, one or more of the distal tips is longitudinally offset to one or more of the other distal tips.

Preferably, one or more of the cantilevered arms comprises a radially inwardly extending portion located proximal of the distal tip of the respective cantilevered arm.

Preferably, the occlusion device comprises an annular support connecting the sleeve and the cantilevered arms, the annular support having a proximal side secured to the distal end of the sleeve and a distal side from which extends the conical array of cantilevered arms.

Preferably, the annular support is configured to substantially isolate or decouple the cantilevered arms from deformations or displacements of the sleeve.

Preferably, the occlusion device comprises an array of resiliently deformable spokes connected between the distal end of the sleeve and the proximal side of the annular support.

Preferably, the annular support is substantially resistant to radial deformation.

Preferably, the annular support defines a region of flexibility about a connection to one or more of the cantilevered arms and/or one or more of the deformable spokes.

Preferably, the proximal and/or distal side of the annular support comprises a wave shaped edge.

Preferably, the sleeve comprises a plurality of interconnected annular sinusoidal ribs defining a reticulated cylindrical sidewall.

Preferably, the cylindrical sidewall comprises a plurality of radially outwardly extending projections.

Preferably, the occlusion device comprises a remotely operable actuator arranged to effect displacement of the sleeve between the expanded and collapsed states.

Preferably, the occlusion device comprises a membrane enclosing the cantilevered arms and at least a portion of the distal end of the sleeve.

Preferably, the membrane surrounds the distal tip of at least one of the cantilevered arms.

Preferably, the membrane is dimensioned to accommodate relative displacement between at least two adjacent cantilevered arms.

Preferably, the occlusion device comprises one or more elements provided at a proximal end of the sleeve and configured to facilitate recapture of the occlusion device.

Preferably, the occlusion device comprises a second haemostatic valve about a proximal end of the sleeve.

Preferably, the second haemostatic valve comprises a conical array of cantilevered arms extending from the proximal end of the sleeve and converging towards tips to define the second haemostatic valve.

Preferably, the first haemostatic valve and/or second haemostatic valve are operable to maintain bidirectional haemostasis regardless of the original direction of flow through a body lumen in which the occlusion device is to be deployed.

According to a further aspect of the present invention there is provided a method of delivering a therapeutic agent into a lumen of the endovascular system comprising: locating into the lumen in a collapsed state an occlusion device comprising a deformable sleeve and a conical array of cantilevered arms extending from a distal end of the sleeve and converging towards distal tips; expanding the sleeve to anchor the occlusion device within the lumen such that the cantilevered arms define a first haemostatic valve; and passing a surgical implement through the array of arms to deliver the therapeutic agent distally of the occlusion device.

Preferably, the method comprises deforming the tips of at least some of the cantilevered arms radially outwardly in response to pressure applied by the surgical implement in order to facilitate passage of the surgical implement.

Preferably, the method comprises biasing at least some of the cantilevered arms against an exterior of the surgical implement in order to maintain haemostasis between the surgical implement and the first haemostatic valve.

Preferably, the method comprises at least partially isolating the haemostatic valve from deformations or displacements of the sleeve by providing the occlusion device with an annular support between the first haemostatic valve and the sleeve.

Preferably, the method comprises passing the surgical implement through the first haemostatic valve in a first axial direction while maintaining haemostasis between the surgical implement and the first haemostatic valve.

Preferably, the method comprises passing the surgical implement through the first haemostatic valve in a second axial direction while maintaining haemostasis between the surgical implement and the first haemostatic valve.

Preferably, the method comprises drawing the surgical implement back through the haemostatic valve and closing the haemostatic valve by the radially inward resilient deformation of the array of cantilevered arms.

Preferably, the method comprises cleaning therapeutic agent from an exterior of the surgical implement while drawing the surgical implement back through the haemostatic valve.

As used herein, the term “surgical implement” is intended to mean any tool or instrument used during deployment/retrieval of the occlusion device and which may pass through the central lumen of the device, including but not limited to a conventional guidewire used to deliver the device to a deployment site and a catheter or the like used to dispense an embolic or the like distally of the device when deployed.

As used herein, the term “annular” is intended to mean any generally circular or ring like element which may be cylindrical or conical in form, which may have a solid or open sidewall, and may have varying levels of stiffness.

As used herein the terms “axial” and “radial” and intended to be with reference to the substantially cylindrical sleeve of the occlusion device which has a longitudinal axis along which the “axial” direction extends and perpendicular to which the “radial” direction extends.

### BRIEF DESCRIPTION OF THE DRAWINGS

The present invention will now be described with reference to the accompanying drawings, in which:

FIG. 1 illustrates a side elevation of an endovascular occlusion device according to an embodiment of the present invention;

FIG. 1a illustrates a schematic representation of the endovascular occlusion device shown in FIG. 1 to illustrate possible angular variation in different sections of the device;

FIG. 2 illustrates a side elevation of a distal end of the occlusion device of FIG. 1;

FIG. 3 illustrates a perspective view of the distal end shown in FIG. 2;

FIG. 4 illustrates a perspective view of the occlusion device of FIGS. 1 to 3;

FIG. 5 illustrates an alternative partial perspective view of the occlusion device of FIGS. 1 to 4;

FIG. 6 illustrates the occlusion device of FIGS. 1 to 5 in an expanded state;

FIG. 7 illustrates the occlusion device of FIG. 6 in a partially collapsed state;

FIG. 8 illustrates the occlusion device of FIGS. 6 and 7 in a fully collapsed state;

FIG. 9 illustrates the occlusion device of FIGS. 1 to 8 and showing a membrane forming part of the device;

FIG. 10 illustrates the occlusion device of FIG. 9 with a distal end open to permit passage of a surgical implement therethrough;

FIG. 11 illustrates the arrangement of FIG. 10 and illustrates a surgical implement passing through the occlusion device;

FIG. 12 illustrates an annular support which forms part of the occlusion device;

FIG. 13 illustrates an alternative form of the annular support shown in FIG. 12;

FIG. 14 illustrates a further alternative form of the annular support shown in FIG. 12;

FIG. 15 illustrates an alternative form of the distal end illustrated in FIG. 2;

FIG. 16 illustrates a further alternative form of the distal end illustrated in FIG. 2;

FIG. 17 illustrates a still further alternative form of the distal end illustrated in FIG. 2;

FIG. 18 illustrates an additional alternative form of the distal end illustrated in FIG. 2;

FIG. 19 illustrates a perspective view of a further alternative form of the distal end illustrated in FIG. 2;

FIG. 20 illustrates a front view of the distal end illustrated in FIG. 19;

FIG. 21 illustrates the occlusion device of FIGS. 1 to 11 provided with a second haemostatic valve about a proximal end of the occlusion device;

FIG. 22 illustrates a side elevation of an endovascular occlusion device according to a second embodiment of the present invention;

FIG. 23 illustrates a side elevation of an endovascular occlusion device according to a third embodiment of the present invention;

FIG. 24 illustrates a side elevation of an endovascular occlusion device according to a fourth embodiment of the present invention;

FIG. 25 illustrates a side elevation of an endovascular occlusion device according to a fifth embodiment of the present invention; and

FIG. 26 illustrates a side elevation of an endovascular occlusion device according to a sixth embodiment of the present invention.

### DETAILED DESCRIPTION OF THE DRAWINGS

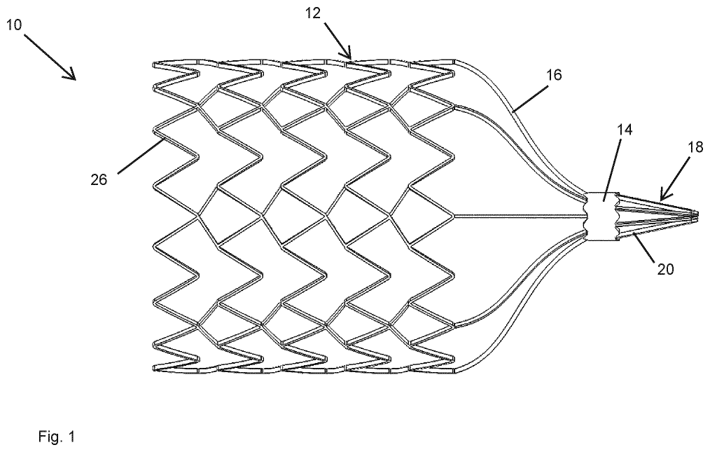

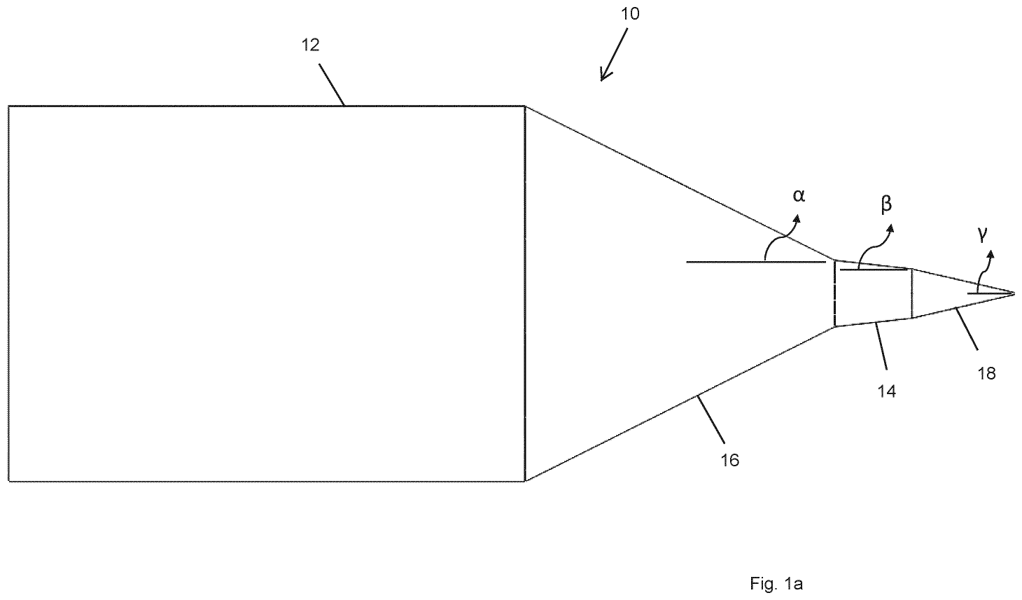

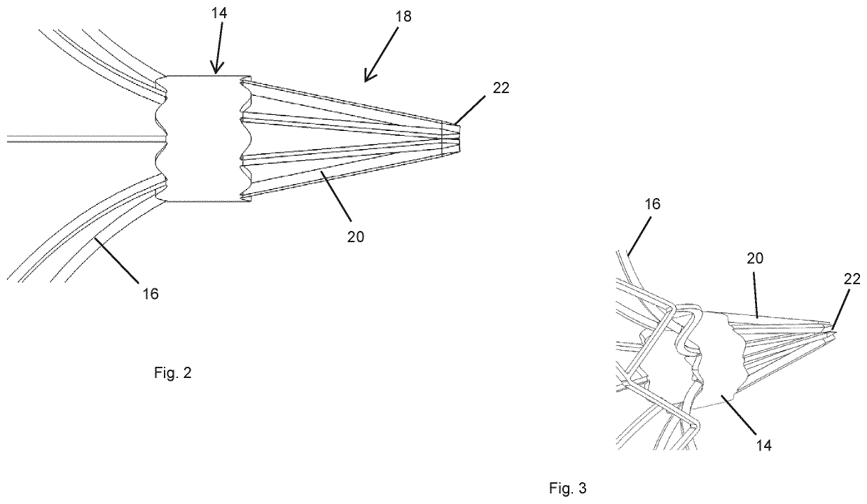

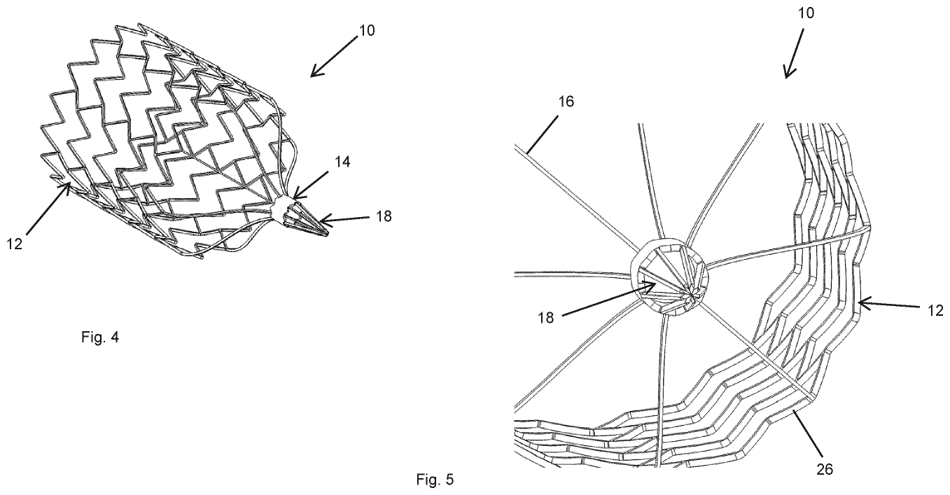

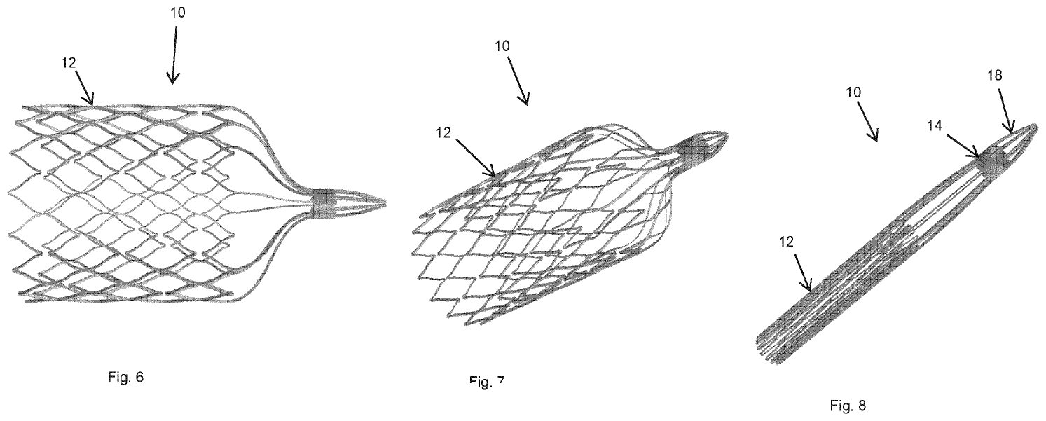

Referring now to FIGS. 1 to 11 of the accompanying drawings there is illustrated an endovascular occlusion device according to an embodiment of the present invention, generally indicated as 10, for use in occluding a lumen of the endovascular system to treat vascular malformations, bleeding vessels or to occlude vessels in order to induce tissue ischemia or necrosis. As described in detail hereinafter the occlusion device 10 functions to obstruct blood flow from the distal part of the device 10 to the proximal side and vice versa, before during and after delivery of an embolic or other therapeutic material at a distal side of the device 10. The occlusion device 10 also induces coagulation.

The occlusion device 10 comprises a sleeve 12 which preferably has an axially symmetric cross section, more preferably of cylindrical form, and is most preferably of mesh like construction. As described in detail hereinafter the sleeve 12 is displaceable between an expanded state as illustrated in FIG. 1, and a collapsed state as illustrated in FIG. 8, and is therefore similar in construction and operation to a vascular stent. In the expanded state the sleeve 12 is radially dimensioned to be engaged and retained against the interior of the wall of the vessel (not shown) in which the endovascular device 10 is to be deployed, and may for example be of a diameter in the range of between 4 mm and 20 mm when expanded. In the expanded state the sleeve 12 defines an interior lumen which provides functionality to the occlusion device 10 as hereinafter described, in particular permitting the passage of a guidewire through the occlusion device 10 to facilitate deployment, and the passage of a catheter to deliver an embolic distally of the occlusion device 10. In the collapsed state the sleeve 12 has a significantly reduced radial dimension in order to be deliverable to the deployment site and/or to allow repositioning or removal of the device 10.

The occlusion device 10 further comprises an annular support 14 axially spaced from a distal side or end of the sleeve 12 and connected to the distal end via an array of spokes 16 which together define a shoulder or transition zone between the sleeve 12 and annular support 14. The annular support 14 and sleeve 12 are preferably but not essentially co-axially arranged. Unlike the sleeve 12 the annular support 14 is preferably substantially resistant to radial deformation, although may have controlled flexibility as described hereinafter. The annular support 14 has a substantially reduced diameter relative to the expanded sleeve 12, and in an exemplary embodiment may have an inner diameter in the range of between 0.5 mm and 4 mm. The annular support 14 may have an outer diameter approximately corresponding to the outer diameter of the sleeve 12 when in the collapsed state, for example as shown in FIG. 8.

The occlusion device 10 further comprises a first haemostatic valve 18 defined by a conical array of cantilevered arms 20 extending from a distal end of the annular support 14 and which converge and terminate at distal tips 22. The cantilevered arms 20 are resiliently deformable or otherwise displaceable in order to allow the passage of a surgical implement such as a catheter or guidewire to pass therebetween, in particular allowing the tips 22 to be reversibly separated from one another through resilient outward deformation of the arms 20. In a preferred arrangement the cantilevered arms 20 are resiliently deformable and/or are provided with a flexible or resiliently deformable connection with the annular support 14. It will however be appreciated that any other mechanism may be employed in order to achieve this functionality, for example one or more hinges or the like may be provided on or about one or more of the cantilevered arms 20. As also described hereinafter the geometry of the arms 20 and tips 22 can be varied in order to alter the operation of the haemostatic valve 18, for example by tapering the arms 20 to define sharp tips 22 which effectively fully converge to create a haemostatic seal, or the tips 22 may be rounded or otherwise profiled to reduce friction with a passing surgical implement such as the guidewire or catheter.

The occlusion device 10 is illustrated schematically in FIG. 1a, highlighting the angular inclination alpha (a) of the transition zone defined by the spokes 16 relative to the longitudinal axis, the angular inclination beta (β) of the annular support 14 and the angular inclination gamma (γ) of the arms 22. Each of these angles may be varied from between 0 and 90 degrees, and for example in FIG. 1 the angle β is zero degrees while in FIG. 1a is greater than zero degrees. The angles selected will have a bearing on the mechanical performance of the various parts of the occlusion device 10, allowing for fine tuning in order to achieve desired performance characteristics.

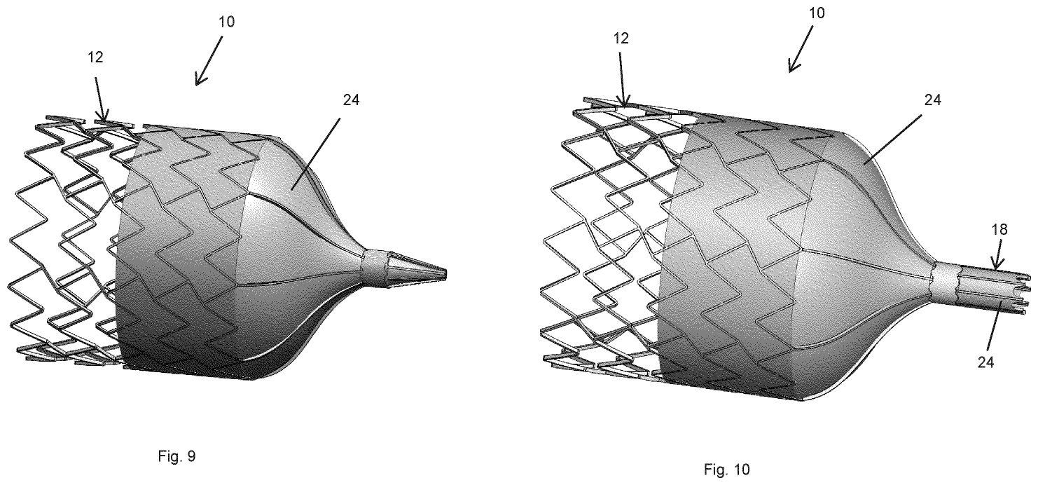

In the first embodiment illustrated the sleeve 12 is formed from a biocompatible self-expanding material such as nitinol and can be at least partially covered with a polymer fluid impermeable membrane 24 as shown in FIGS. 9 to 11, such as polytetrafluoroethylene (PTFE), woven fibres (such as Dacron) or other elastomeric materials (silicone, polyurethane or their co-polymers). The membrane 24 is arranged to cover at least a distal end of the sleeve 12 and to extend over the spokes 16 and the haemostatic valve 18 in order to provide a seal to prevent fluid flow through the occlusion device 10 in either direction. The sleeve 12 is defined by a plurality of annular rings 26 arranged axially adjacent and connected to one another, each ring 26 comprising a sinusoidal or wave like element which facilitates displacement of the sleeve 12 between the expanded and collapsed states through relative articulation and/or flexing of adjacent sections to provide a concertina type action. The number of rings 26 may be varied as required, for example to vary the overall length of the sleeve 12. In the embodiment illustrated each ring 26 defines sixteen peaks and troughs, and adjacent rings 26 are secured to one another via a plurality of struts 28 which extend between aligned peaks of the adjacent rings 26. In one embodiment this arrangement will create an open cell configuration such that every two cells create a closed cell. The open cell design will encourage intimal hyperplasia and allow vascular endothelium to project through the sleeve 12. This configuration will allow tissue to prolapse between the interstices of the sleeve 12, thus providing an anti-migration anchoring feature preventing unintended movement of the occlusion device 10 following deployment. In an exemplary but non-limiting embodiment the defining element of the rings 26 is 50˜400 μm in width and 50˜400 μm in thickness.

The sleeve 12 can be crimped onto a delivery system (not shown) prior to delivery and deployment within the target vessel. At a neutral position once out of the delivery system the sleeve 12 will expand to the required size (for example between 4 mm and 20 mm). Once deployed in the vessel, the outer diameter of the sleeve 12 will reduce from the heat set diameter to a reduced diameter due to the radial resistive force applied by the vessel wall and as a result of the resilience of the sleeve 12 will tend to exhibit a chronic outward force on the wall such as to protect against migration. The polymer membrane 24 is positioned and arranged such as not to inhibit this mechanical action, for example by not covering the entire length of the sleeve 12.

It will be understood that the number, dimensions and exact geometry of the rings 26 may be varied as required. For example one or more of the peaks may be raised out of the cylindrical surface of the sleeve 12 to provide additional anchoring into the blood vessel. Additionally or alternatively the cross-sectional profile of the sleeve 12 may be modified to decrease the effective radius of curvature at one or more locations on the surface of the sleeve 12 to enhance stress concentration and anchoring in the wall of the vessel. As a further modification alternative rings 26 may be heat-set at different diameters to increase stress concentrations and anchoring on deployment of the occlusion device 10.

The spokes 16 provide a transition between the relatively large diameter and flexible sleeve 12 (in the deployed or expanded state) and the relatively stiff or incompressible annular support 14. The occlusion device 10 preferably comprises between four and eight of the spokes 16 and which may have a width of between 50˜400 μm and a thickness of between 50-400 μm and are covered by the membrane 24. The spokes 16 may be of a different cross sectional profile to the rings 26 and the geometry thereof may be selected to at least partially isolate the displacement of the sleeve 12 from the remainder of the occlusion device 10, in particular the annular support 14 and haemostatic valve 18.

The spokes 16 may be formed of the same biocompatible material as the rings 26 of the sleeve 12, and in the embodiment illustrated have a sinusoidal profile in the radial direction. This profile facilitates the controlled deformation of the spokes 16 during displacement of the sleeve 12 between the expanded and collapsed states, again to minimise the transmission of any deformations or movements of the sleeve 12 to the remainder of the occlusion device 10 during use. The sinusoidal profile of the spokes 16 retains the annular support 14 and haemostatic valve 18 centred within the vessel and prevent against device rotation, deforming or bending, regardless of the diameter of the vessel. This is an important aspect of the operation of the occlusion device 10, keeping the central lumen of the annular support 14 and haemostatic valve 18 axially aligned to allows a vascular catheter to pass through the central lumen of the occlusion device 10.

It will of course be understood that the number and configuration of the spokes 16 may be varied. The spokes 16 may for example be shaped to define a curve in the axial direction, such that an end of a spoke 18 connected to the sleeve 12 may be axially offset to an opposed end of the spoke 18 connected to the annular support 14. Such a profile may provide flexibility in a rotational displacement of the sleeve 12, further isolating the said rotational displacement from the annular support 14 and haemostatic valve 18. There may also be provided a number of radial elements or spars (not shown) connected between two or more adjacent spokes 16. These would provide additional rotational stability during use. Additionally or alternatively one or more of the spokes 16 and/or connecting struts (not shown) may have a wave like profile similar to the rings 26 to facilitate bending and therefore absorb distortions being transmitted from the sleeve 12.

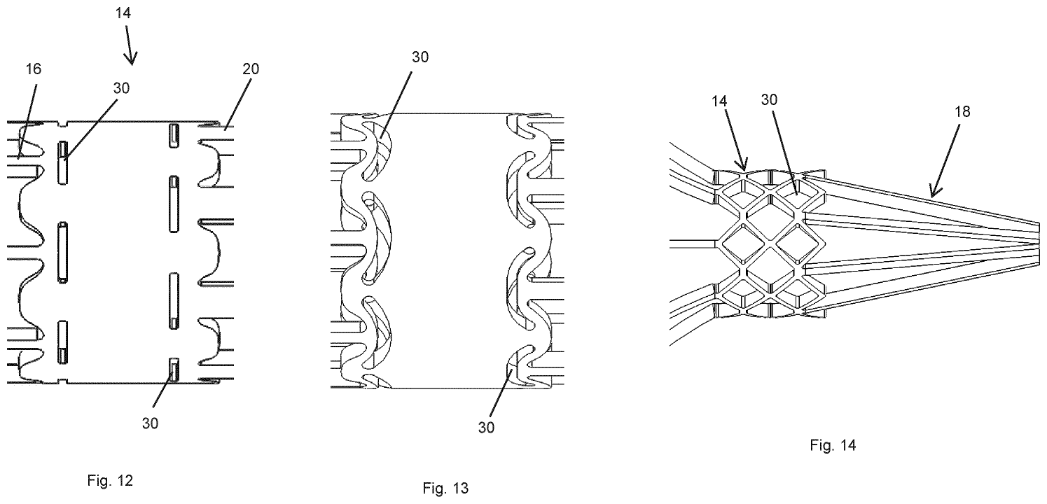

Referring in particular to FIGS. 12 to 14 the annular support 14 is shown in isolation in a number of alternative configurations. The annular support 14 serves two main functions, the first being the above noted isolation of movement or distortions of the sleeve 12 from the haemostatic valve 18. The second is the provision of a stable platform or base from which the cantilever arms 20 of the haemostatic valve 18 extend and are displaceable relative to in order to accommodate the passage of a surgical implement while providing a haemostatic seal at the distal end of the occlusion device 10. As noted above the annular support 14 is preferably but not essentially resistant to radial compression in order to resist deformation transmitted from the sleeve 12 during use, thereby providing a stable platform for the cantilever arms 20 ensuring consistent operation therefore during use of the occlusion device 10. The annular support 14 may define a substantially solid sidewall as shown in FIGS. 12 and 13 but may equally be cellular or reticulated as shown for example in FIG. 14. The annular support 14 may offer a low level of compressibility or crimpablity and thereby undergo expansion during deployment. It is however also envisaged that the occlusion device 10 may be configured without the annular support 14, whereby the array of cantilevered arms 20 would extend directly from the spokes 16, although such arrangements would not provide the above described isolation of the haemostatic valve 18 from the sleeve 12.

Referring to FIG. 12 the annular support 14 may be provided with holes 30 adjacent the proximal end thereof at which the spokes 16 connect. These holes 30 provide a locally increased level of flexibility thus reducing stress concentration at those areas, which effectively act as a hinge between the spoke 18 and the annular support 14. Corresponding holes 30 are provided adjacent the distal end in order to provide the same functionality with respect to the cantilevered arms 20. The annular support 14 is preferably also provided with a crenelated or zigzag proximal and distal edge. The spokes 16 and cantilever arms 20 are connected to a respective valley of the respective zigzag edge. This serves to shorten the overall length of the occlusion device 10. The profiled edges additionally provide flexibility and protect against loop/rotational forces potentially transmitted by the spokes 16 and/or cantilevered arms 20. The valleys or troughs of the distal edge are preferably offset or out of phase with the valleys/troughs of the proximal edge. This acts to further avoid the transfer of displacement or distortions between the spokes 16 and arms 20, again further stabilising the occlusion device 10 to ensure consistent performance during use. In the arrangement of FIG. 14 the holes 30 are configured to define a lattice or open frame type arrangement for the annular support 14. It will be appreciated that more or less of the annular rows of the framework may be employed in order to achieve the desired mechanical properties, varying the overall or localised flexibility of the support 14.

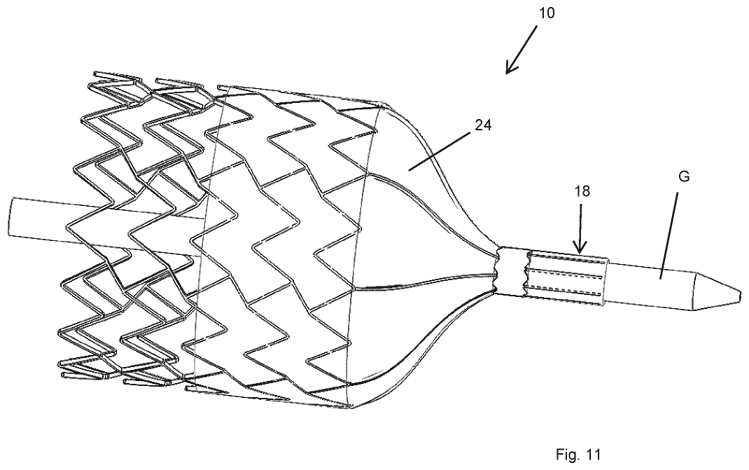

Extending distally from the annular support 14 is the haemostatic valve 18 which is preferably comprised of between four and eight of the cantilevered arms 20. In an exemplary embodiment the cantilevered arms 20 are between 50˜400 μm in width (circumferential dimension) and between 50˜400 μm in thickness (radial dimension). The cantilevered arms 20 are preferably disposed at an angle to a central or longitudinal axis of the occlusion device 10 of between 20˜60 degrees. The cantilevered arms 20 are preferably formed from a biocompatible and resiliently deformable material, and may be formed of the same material as the sleeve 12 and/or the spokes 18. The cantilevered arms 20 are configured to converge at the distal tips 22 such as to provide a closed end which, in particular when the valve 18 is wrapped by the membrane 24, establishes a haemostatic seal. However the cantilevered arms 20 are resiliently deformable and thus the tips 22 may be forced away from one another to provide a cannulation/through-lumen to permit the passage of a surgical implement through the haemostatic valve 18. Additionally, when a guidewire G (see FIG. 11) or a vascular catheter is pushed through the lumen of the occlusion device 10 the cantilevered arms 20 will deform to open the haemostatic valve 18 allowing the guidewire G or catheter to pass through the lumen and access a distal side of the occlusion device 10 within the vessel in which the device 10 is deployed. The tips 22 of the cantilevered arms 20 may be profiled or otherwise configured to generate low friction with the guidewire/catheter allowing the cantilevered arms 20 to be easily opened upon introducing the guidewire/catheter. The membrane 24 is dimensioned, in particular between adjacent cantilevered arms 20, to permit this relative movement during opening and closing of the haemostatic valve 18 and thus ensure a seal is established around the guidewire or catheter preventing fluid leakage across the occlusion device 10.

The cantilevered arms 20 may be configured to tune the flexibility and movement thereof during use. For example the cross section of the cantilevered arms 20 is preferably square or rectangular at the connection to the annular support 14 and to then taper in thickness towards the distal tips 22. A tapered cross section provides sufficient space for the cantilevered arms 20 to converge at an apex defining the most distal part of the occlusion device 10. Such a configuration established a haemostatic seal. The profile of the cantilevered arms 20 and/or tips 22 may be varied to provide certain functionality. For example the cross section of the cantilevered arms 20 tapers with a sharp distal tip 22 while in an alternative configuration the distal tips 22 have a rounded taper to provide lower friction between the tips 22 and the guidewire/catheter. It is preferred that the aspect ratio of the cantilevered arms 20 is selected to minimise the risk of off-axis bending during displacement of the cantilevered arms 20, which could compromise the function and symmetry of the haemostatic valve 18.

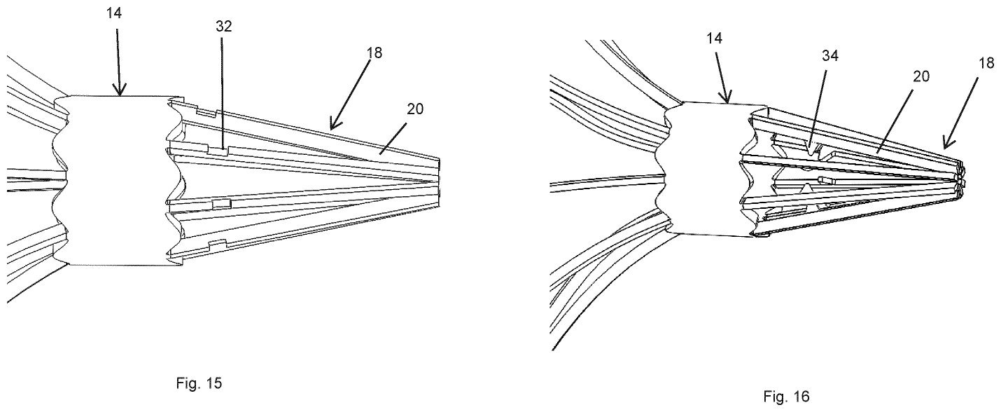

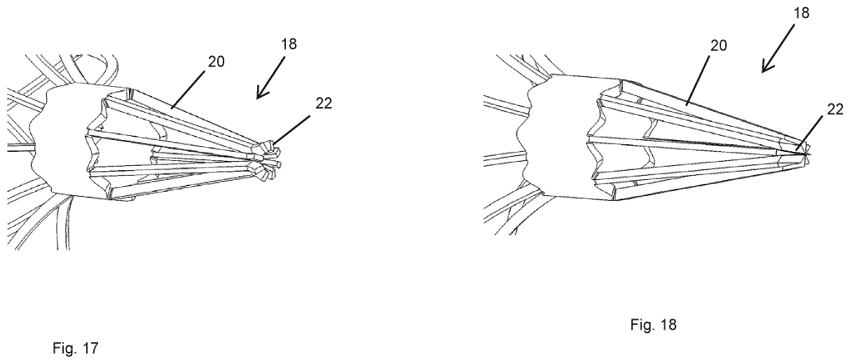

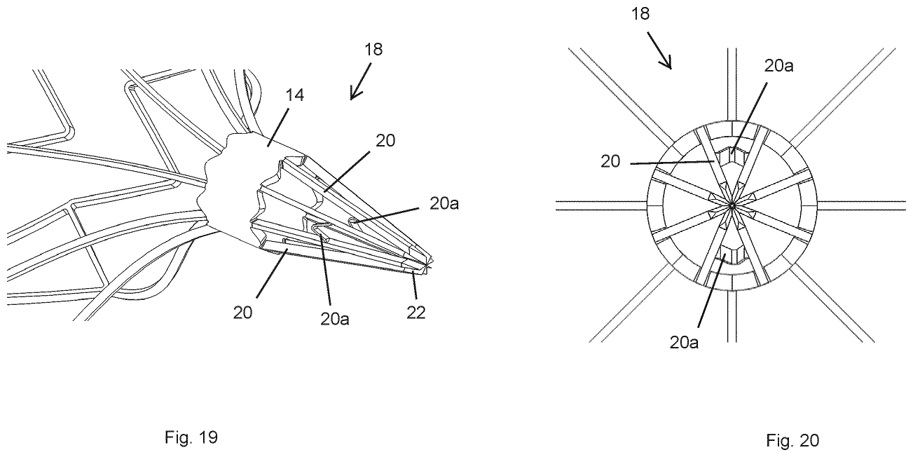

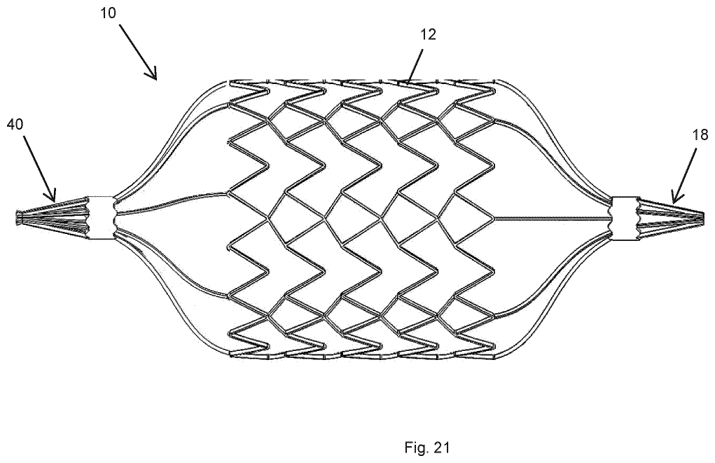

Referring to FIG. 15 the cantilevered arms 20 may be provided with a notch 32 or equivalent local reduction in thickness in order to define a live hinge or area of increased flexibility to allow the cantilevered arms 20 to be deformed radially outward to facilitate the passage of a guidewire/catheter. FIG. 16 illustrates a modification to the cantilevered arms 20 to provide a radially inwardly extending abutment 34 which will be contacted by an advancing guidewire/catheter in order to force the cantilevered arms 20 radially outwardly. Similar functionality may be achieved but provided the cantilevered arms 20 with a curved or bowed shape in the radial direction, such that the cantilevered arms 20 converge towards one another along a curve path. When the guidewire/catheter passes through the haemostatic valve 18 it will first engage with those curved or converging portions, opening the haemostatic valve 18 and eliminating any engagement between the distal tips 22 and the guidewire/Catheter. This will avoid potential damage of the guidewire/catheter and will also prevent inward bending of the cantilevered arms 20 upon withdrawing the guidewire/catheter which might otherwise occur as a result of excessive friction therebetween. A similar arrangement is illustrated in FIG. 17, although the cantilevered arms 20 are not curved but rather define a convergent/divergent configuration. FIG. 18 illustrated the cantilevered arms 20 when the distal tips 22 are sharped into fine points to ensure complete convergence. In FIGS. 19 and 20 two opposed pairs of the cantilevered arms 20 are connected together via a respective flexible coupling 20a positioned at a location between the annular support 14 and the distal tip 22, or at the distal tip 22, in order to again tune the mechanical performance of a number of the cantilevered arms 20. It will be appreciated that a greater or lesser amount of the cantilevered arms 20 may be secured together in this manner.

The haemostatic valve 18 may also be configured such that every second cantilevered arm 20 is reduced in length and/or oriented further radially inward to cause the membrane 24 between each of the adjacent two longer cantilevered arms 20 to fold inward at the distal part, providing full sealing at the distal tips 22.

A portion of fabric material such as Dacron or other polymer/fibre can be attached at the distal tip 22 of the cantilevered arms 20 to act as a prothrombotic fibre but additional to act as a wiper seal or brush when the embolic injecting catheter is withdrawn, thereby cleaning embolic liquid or other liquid/particles from the exterior surface of the injecting catheter. This will protect against inadvertent reflux of glue or other material proximally as the catheter is withdrawn back through the lumen/channel of the occlusion device 10. The above modifications may be combined in various permutations and combinations with any of the embodiments disclosed herein in order to achieve the necessary performance or use characteristics.

Referring now to FIG. 21 the occlusion device 10 is illustrated in which a second haemostatic valve 40 of the same configuration and operation as the first haemostatic valve 18 is provided, but located at the opposite end of the sleeve 12, and thus at a proximal side of the occlusion device 10. This arrangement provides an additional level of haemostatic sealing and facilitates the recapture of the occlusion device 10 after initial deployments from the delivery sheath or system (not shown) should the operator believe that the deployed position is suboptimal. A similar tapered geometry could also be used to facilitate device recapture without incorporating the second haemostatic valve 40.

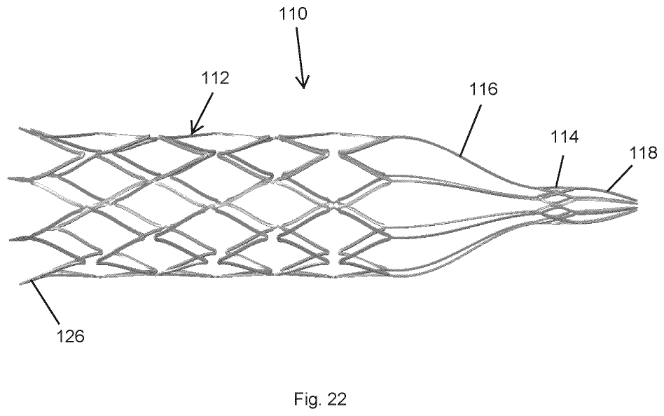

Turning now to FIG. 22 there is illustrated a second embodiment of an occlusion device according to the present invention, generally indicated as 110, for use in occluding a lumen of the endovascular system to treat vascular malformations, bleeding vessels or to occlude vessels in order to induce tissue ischemia or necrosis. In this second embodiment like components have been accorded like reference numerals and unless otherwise stated perform a like function. The occlusion device 110 again comprises a collapsible sleeve 112, an annular support 114, shoulder or transition zone defined by spokes 116, and a haemostatic valve 118. A proximal end of the sleeve 112, as defined by one of an array of annular rings 126, is flared radially outwardly such that the ring 26 forms a circular array of protrusions which in use will engage the wall of the vessel into which the device 110 is deployed in order to provided additional anchorage for retaining the device 110 in position.

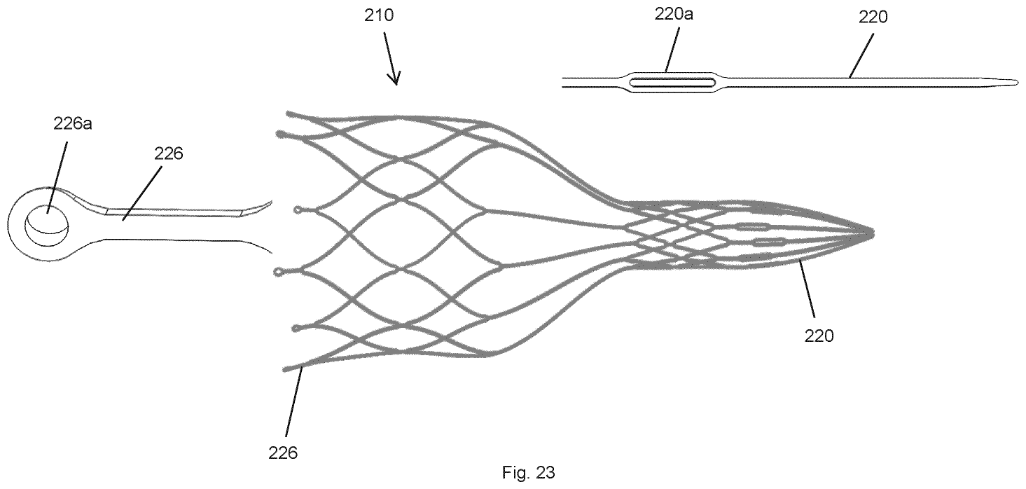

FIG. 23 illustrates a third embodiment of an occlusion device according to the present invention, generally indicated as 210. In this third embodiment like components have been accorded like reference numerals and unless otherwise stated perform a like function. The occlusion device 210 again comprises a flared proximal end defined by one of an array of annular rings 226 forms a circular array of protrusions which in use will engage the wall of the vessel into which the device 210 is deployed in order to provided additional anchorage. Each of the protrusions, one shown enlarged on the left hand side of FIG. 23, defined an aperture 226a which can incorporate a radiopaque marker in order to allow the proximal end of the occlusion device 210 to be imaged during deployment or subsequent procedure. Similarly one or more cantilevered arms 220 may define an opening 220a which can also incorporate a radiopaque marker to allow the distal end of the device 210 to be imaged, with one such arm 220 shown enlarged on the right had side of FIG. 23.

FIG. 24 illustrates a fourth embodiment of an occlusion device according to the present invention, generally indicated as 310. In this fourth embodiment like components have been accorded like reference numerals and unless otherwise stated perform a like function. The occlusion device comprises a collapsible sleeve 312, an annular support 314, transition zone or shoulder defined by spokes 316 and a haemostatic valve defined by cantilevered arms 320. Unlike in previous embodiments the annular support 314 is positively inclined to the longitudinal axis, such as to have a positive angle beta as detailed above with reference to FIG. 1a. The annular support 314 is also of a simple lattice or framework configuration, having a simple zigzag form, with the arms 320 extending form the peaks of these zigzags while the spokes 316 connect with the troughs of the zigzags. This arrangement allows the overall length of the occlusion device 310 to be shorter and the isolating functionality of the support 314 to be tuned.

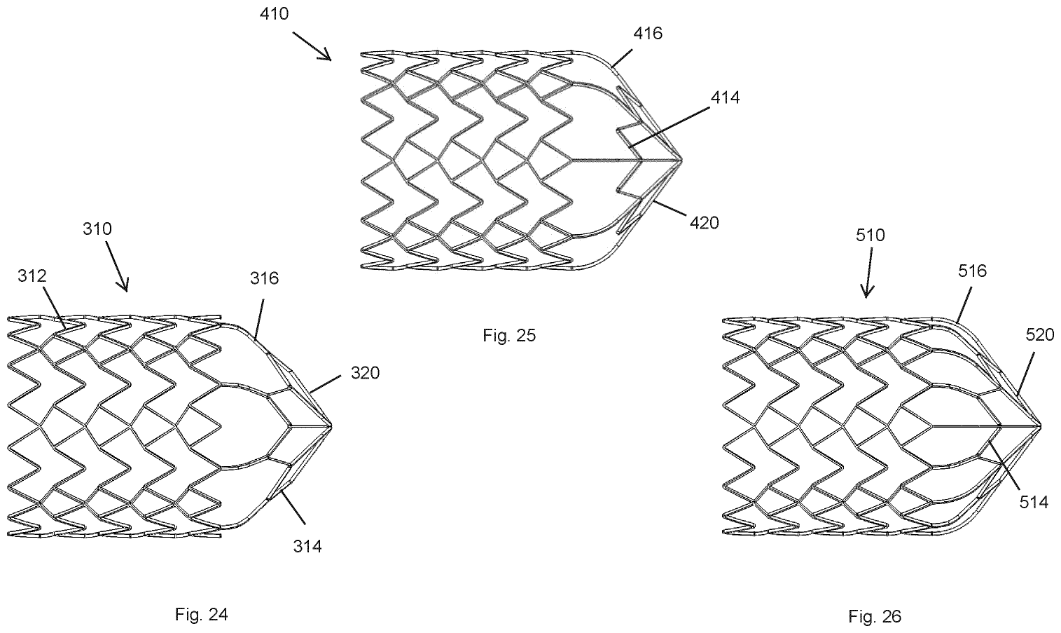

FIG. 25 illustrates a fifth embodiment of an occlusion device, generally indicated as 410 and of similar constructions to the fourth embodiment, but with spokes 416 and cantilevered arms 420 connected to the same location on an annular support 414, namely peaks of the zigzag form of the support 414, again provided an alternative tune to the flexibility of the components parts of the device 310.

FIG. 25 illustrates a sixth embodiment of an occlusion device, generally indicated as 510 and of similar constructions to the fourth, but with a higher count of spokes 516 and which are therefore connected to both peaks and troughs of a wave or zigzag formed annular support 514. An array of cantilevered arms 520 for a haemostatic valve, one arm 520 extending from each peak of the support 514.

In use the occlusion device 10; 110; 210; 310; 410; 510 is initially provided in the collapsed state as shown in FIG. 8, and preferably for delivery through a vascular catheter or sheath or bespoke delivery system (not shown) and most likely along a preinstalled guidewire G over which the occlusion device 10; 110; 210; 310; 410; 510 can pass as hereinbefore described. On reaching the deployment site the occlusion device 10; 110; 210; 310; 410; 510 is displaced outwardly from the vascular catheter. The occlusion device 10; 110; 210; 310; 410; 510 may comprise a remotely operable actuator (not shown) which may be triggered to allow the sleeve to expand outwardly into the deployed or expanded state. In the absence of such an actuator the sleeve will simply expand as soon as it is displaced out of the vascular catheter. The occlusion device 10; 110; 210; 310; 410; 510 will then press against the wall of the blood vessel in order to anchor in place.

Following deployment of the occlusion device 10; 110; 210; 310; 410; 510, the relative rigidity of the annular support 14; 114; 214; 314; 414; 514 can be utilised as a backstop by the delivery catheter/sheath (not shown) to help disengage an introducer/pusher (not shown) from the deployed occlusion device 10; 110; 210; 310; 410; 510.

At this point the vascular catheter may be withdrawn, or may be used to advance an injection catheter to the deployment site. The injection catheter is then advanced axially into the lumen of the occlusion device 10; 110; 210; 310; 410; 510 and will then engage the interior surface of the cantilever arms 20;, forcing the cantilever arms 20 to resiliently deform radially outward as shown in FIGS. 10 and 11 in order to permit the passage of the injection catheter. The resilience of the cantilevered arms 20 ensure that they remain engaged against the exterior of the injection catheter and, in combination with the surrounding membrane 24 (if provided), prevent any fluid flow through the occlusion device 10; 110; 210; 310; 410; 510 in either direction.

Once the tip of the injection catheter has been located at the appropriate position distally of the occlusion device 10; 110; 210; 310; 410; 510 a liquid embolic or other substance or therapeutic material may then be injected from the catheter into the vessel. The injection catheter can then be removed, following the cantilevered arms 20 will deformed back together to establish a suitable seal to prevent reflux of the embolic or other substance. It will therefore be appreciated that it is the resilience and orientation the cantilevered arms 20 that ensures haemostasis and as a result the occlusion device 10; 110; 210; 310; 410; 510 does not rely on blood pressure within the vessel to close the haemostatic valve 18; 118. As a result the occlusion device 10; 110; 210; 310; 410; 510 provides haemostasis regardless of the direction of blood or fluid flow from proximal to distal or vice versa.

The occlusion device 10; 110; 210; 310; 410; 510 of the present invention thus allows immediate proximal permanent embolization with combined occlusion of the vessel. Repositioning and/or withdrawal and replacement of the occlusion device 10; 110; 210; 310; 410; 510 may also be possible after positioning prior to final release and deployment. Unlike current coils or vascular plugs, a catheter can then pass through the occlusion device 10; 110; 210; 310; 410; 510 to access a distal side thereof. This allows safe effective distal small vessel embolization with a liquid embolic or small particle without the risk of the liquid or small particles refluxing or flowing proximally. Alternatively, it allows high dose distal drug infusion or small particle radiation treatment through the proximally occluded vessel without risk of reflux. The device 10; 110; 210; 310; 41; 510 allows various embolics including but not limited to microparticles to be delivered distal to the deployed device 10; 110; 210; 310; 410; 510 through commercially available vascular catheter and micro-catheters (not shown) independent of the delivery system (not shown) used to deploy the device 10; 110; 210; 310; 410; 510. Such catheters and micro-catheters can be manipulated and directed distal to the device 10; 110; 210; 310; 410; 510 allowing more distal and targeted delivery of these forms of embolics.

1. An endovascular occlusion device comprising a deformable sleeve displaceable between a collapsed state and an expanded state; and a conical array of cantilevered arms extending from a distal end of the sleeve and converging towards distal tips such as to define a first haemostatic valve about the distal end of the sleeve.

2. An endovascular occlusion device according to claim 1 in which the resilience of the array of cantilevered arms acts to bias the first haemostatic valve into a closed state.

3. An endovascular occlusion device according to claim 1 in which the array of cantilevered arms are sufficiently resiliently deformable to permit the tips to separate radially to facilitate the passage of a surgical implement.

4. An endovascular occlusion device according to claim 1 in which the array of cantilevered arms are sufficiently resiliently deformable to exert a radially compressive force on a surgical implement projecting through the first haemostatic valve sufficient to maintain haemostasis between the implement and the first haemostatic valve.

5. An endovascular occlusion device according to claim 1 in which one or more of the cantilevered arms vary in cross sectional area along the length of the cantilevered arm.

6. An endovascular occlusion device according to claim 1 in which one or more of the cantilevered arms comprises a live hinge.

7. An endovascular occlusion device according to claim 1 in which the distal tips of the cantilevered arms are configured to cooperate with one another in order to establish a haemostatic seal at the distal tips.

8. An endovascular occlusion device according to claim 1 in which one or more of the distal tips is longitudinally offset to one or more of the other distal tips.

9. An endovascular occlusion device according to claim 1 in which one or more of the cantilevered arms comprises a radially inwardly extending portion located proximal of the distal tip of the respective cantilevered arm.

10. An endovascular occlusion device according to claim 1 comprising an annular support connecting the sleeve and the cantilevered arms, the annular support having a proximal side secured to the distal end of the sleeve and a distal side from which extends the conical array of cantilevered arms.

11. An endovascular occlusion device according to claim 10 in which the annular support is configured to substantially isolate or decouple the cantilevered arms from deformations or displacements of the sleeve.

12. An endovascular occlusion device according to claim 10 comprising an array of resiliently deformable spokes connected between the distal end of the sleeve and the proximal side of the annular support.

13. An endovascular occlusion device according to claim 10 in which the annular support is substantially resistant to radial deformation.

14. An endovascular occlusion device according to claim 10 in which the annular support defines a region of flexibility about a connection to one or more of the cantilevered arms and/or one or more of the deformable spokes.

15. An endovascular occlusion device according to claim 10 in which the proximal and/or distal side of the annular support comprises a wave shaped edge.

16. An endovascular occlusion device according to claim 1 in which the sleeve comprises a plurality of interconnected annular sinusoidal ribs defining a reticulated cylindrical sidewall.

17. An endovascular occlusion device according to claim 16 in which the cylindrical sidewall comprises a plurality of radially outwardly extending projections.

18. An endovascular occlusion device according to claim 1 comprising a remotely operable actuator arranged to effect displacement of the sleeve between the expanded and collapsed states.

19. An endovascular occlusion device according to claim 1 comprising a membrane enclosing the cantilevered arms and at least a portion of the distal end of the sleeve.

20. An endovascular occlusion device according to claim 19 in which the membrane surrounds the distal tip of at least one of the cantilevered arms.

21. An endovascular occlusion device according to claim 19 which the membrane is dimensioned to accommodate relative displacement between at least two adjacent cantilevered arms.

22. An endovascular occlusion device according to claim 1 comprising one or more elements provided at a proximal end of the sleeve and configured to facilitate recapture of the occlusion device.

23. An endovascular occlusion device according to claim 1 comprising a second haemostatic valve about a proximal end of the sleeve.

24. An endovascular occlusion device according to claim 23 in which the second haemostatic valve comprises a conical array of cantilevered arms extending from the proximal end of the sleeve and converging towards tips to define the second haemostatic valve.

25. An endovascular occlusion device according to claim 1 in which the first haemostatic valve and/or second haemostatic valve are operable to maintain haemostasis regardless of the original direction of flow through a body lumen in which the occlusion device is to be deployed.

26. A method of delivering a therapeutic agent into a lumen of the endovascular system comprising: locating into the lumen in a collapsed state an occlusion device comprising a deformable sleeve and a conical array of cantilevered arms extending from a distal end of the sleeve and converging towards distal tips; expanding the sleeve to anchor the occlusion device within the lumen such that the cantilevered arms define a first haemostatic valve; and passing a surgical implement through the array of arms to deliver the therapeutic agent distally of the occlusion device.

27. A method according to claim 26 comprising deforming the tips of at least some of the cantilevered arms radially outwardly in response to pressure applied by the surgical implement in order to facilitate passage of the surgical implement.

28. A method according to claim 26 comprising biasing at least some of the cantilevered arms against an exterior of the surgical implement in order to maintain haemostasis between the surgical implement and the first haemostatic valve.

29. A method according to claim 26 comprising at least partially isolating the haemostatic valve from deformations or displacements of the sleeve by providing the occlusion device with an annular support between the first haemostatic valve and the sleeve.

30. A method according to claim 26 comprising passing the surgical implement through the first haemostatic valve in a first axial direction while maintaining haemostasis between the surgical implement and the first haemostatic valve.

31. A method according to claim 26 comprising passing the surgical implement through the first haemostatic valve in a second axial direction while maintaining haemostasis between the surgical implement and the first haemostatic valve.

32. A method according to claim 26 comprising drawing the surgical implement back through the haemostatic valve and closing the haemostatic valve by the radially inward resilient deformation of the array of cantilevered arms.

33. A method according to claim 26 comprising manipulating the surgical implement in multiple non-axial directions distal to the occlusion device in order to achieve optimal therapeutic delivery of the therapeutic agent.

34. A method according to claim 32 comprising cleaning therapeutic agent from an exterior of the surgical implement while drawing the surgical implement back through the haemostatic valve.
